(19) United States
(12) Patent Application Publication
(10) Pub. No.: US 2019/0223578 A1
(43) Pub. Date: Jul. 25, 2019

(54) Thermoformable Retainer For Facemasks

(71) Applicant: Alps South, LLC, St. Petersburg, FL (US)

(72) Inventor: Aldo Laghi, Pinellas Park, FL (US)

(21) Appl. No.: 16/008,285

(22) Filed: Jun. 14, 2018

Related U.S. Application Data
(60) Provisional application No. 62/619,936, filed on Jan. 22, 2018.

(51) Int. Cl. A45D 44/00; A61K 8/02; B29C 51/00; B29C 51/14

(52) U.S. Cl. CPC A45D 44/002; A61K 8/0212; B29L 2031/718; B29C 51/145; B29C 51/002

(57) ABSTRACT

A cosmetic or therapeutic mask retainer which is thermoformable so as to allow the facemask to remain in position on a user's face when in use. Specifically, the present invention relates to a multi-layered retainer for therapeutic or cosmetic purposes comprising a gelatinous elastomer layer, optional internal fabric layer, a thermoformable plastic layer, and a fabric strap layer. The thermoformable plastic is preferably a plastic that can be heated to temperatures that allow it to be molded but also so that human skin can withstand the heat, such as EVA or PCL. The facemask can be one, two, three, or four components when in use and may be formed by creating a variety of composites between the various layers.

### CROSS-REFERENCE TO RELATED APPLICATIONS

This application claims the benefit of pending provisional application 62/619,936 filed Jan. 22, 2018, the disclosure of which is incorporated by reference herein.

### TECHNICAL FIELD

This disclosure relates to a thermoformable support for facemasks. More specifically, this disclosure relates to a device that may be customized to unique facial geometry and may be used in conjunction with cosmetic and/or therapeutic masks, ointments, creams, or other materials or substances that require more substantial contact or pressure to the user's face.

### BACKGROUND OF THE INVENTION

A variety of masks exist that include ingredients, active and inactive, for the purposes of exfoliating, softening, cleansing, whitening, or other therapeutic and/or cosmetic purposes. Such masks often have backing material made of paper or are of knit or non-woven construction. The application for these masks requires the user to unfold and place against their face with careful attention to the positioning and to ensure contact. It should be noted that the backing materials mentioned are two dimensional and are then placed onto a face which is three dimensional. This makes full contact challenging. Many mask backing materials are provided with an array of slits in and around the mask to facilitate the contouring of the two dimensional mask to the three dimensional face.

Another type of mask exists that is comprised of a hydrogel. This type of mask, also in a two-dimensional configuration, has the inherent difficulties conforming to the three dimensional contours of a user's face.

Still another type of masks exists comprised of a thermoplastic elastomer for purposes of providing active ingredients to the user's skin and/or thermal therapy. In one embodiment the material is a phase change material. While such material possesses favorable stretch characteristics the inherent issue remains of applying the mask evenly throughout the various contours of the user's face.

Therefore, it is an object of the present invention to provide a device that is thermoformable and may be customized to the unique geometry of the user's face.

It is another object of the present invention that the device maintains the shape of the user's face upon cooling below the glass transition point of the material.

It is another object of the present invention that the thermoformable device retains a mask, cream, ointment or other cosmetic or therapeutic material or medical device for application to the face hereinafter “cosmetic or therapeutic mask” for application to the user's face.

It is another object of the present invention to combine the thermoformable retainer of this invention with one or more cosmetic or therapeutic mask(s) by bonding, adhering, or fastening such devices to the interior surface of the retainer.

It is another object of the present invention to combine the thermoformable retainer of this invention with one or more components of a CPAP mask such as the silicone layer thereby creating a better seal around the periphery of the seal layer of the mask.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to a facemask retainer which is thermoformable so as to allow the cosmetic or therapeutic mask to remain in position on a user's face when in use. Specifically, the present invention relates to a multi-layered retainer for a cosmetic or therapeutic mask comprising a gelatinous elastomer layer, optional internal fabric layer, a thermoformable plastic layer, and a fabric strap layer. The thermoformable plastic is preferably a plastic that can be heated to temperatures that allow it to be molded but also so that human skin can withstand the heat, such as EVA or PCL. The cosmetic or therapeutic mask retainer can be one, two, three, or four components when in use and may be formed by creating a variety of composites between the various layers.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a perspective view of a two-piece embodiment of the present invention.

FIGS. 2 and 3 are perspective views of another two-piece embodiment of the present invention in which the thermoformable layer has a bonded layer of fabric.

FIG. 3A is a section view of the embodiment shown in FIG. 3.

FIGS. 4 and 5 are perspective views of one-piece embodiment of the present invention in which the strapped fabric is bonded to the thermoformable layer.

FIG. 5A is a section view of the embodiment shown in FIG. 5.

FIGS. 6 and 7 are perspective views of a one-piece embodiment of the present invention in which the thermoformable layer has one layer of strapped fabric bonded to one side and one layer of strapless fabric bonded to the other side.

FIG. 7A is a section view of the embodiment shown in FIG. 7.

FIGS. 8 and 9 are perspective views of a one-piece embodiment of the present invention in which the thermoformable layer is bonded to the strapped fabric on one side and strapless fabric is bonded onto the other side. In this embodiment the cosmetic or therapeutic mask is affixed to the other side of the strapless fabric.

FIG. 9A is a section view of an embodiment shown in FIG. 9.

### DETAILED DESCRIPTION OF THE DRAWINGS

The following description is of the best mode presently contemplated for carrying out the invention. This description is not to be taken in a limiting sense, but is made merely for the purpose of describing one or more preferred embodiments of the invention. The scope of the invention should be determined with reference to the claims. The various components of the present invention, and the manner in which they interrelate, are described in greater detail hereinafter.

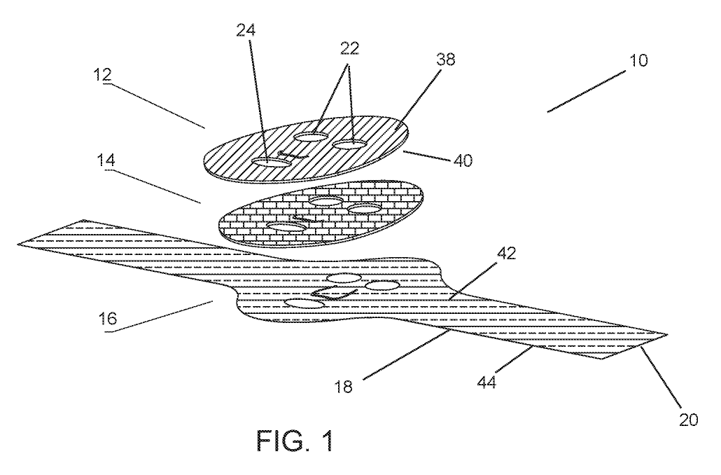

As illustrated in FIG. 1, the present invention relates to a thermoformable retainer for a cosmetic or therapeutic mask 10 in which heat is applied to a thermoformable layer 14 that is comprised of a polymer such as ethyl vinyl acetate (EVA) or polycaprolactone (PCL) or any admixture that is thermoformable at or below 60° C. After being heated to the glass transition temperature, the thermoformable layer 14 is applied to the user's face, depressed into the various contours, and allowed to cool until rigid, thus maintaining the geometry from which it was applied.

After the thermoformable layer has cooled, the mask or other cosmetic or therapeutic device 12 is applied to the inside surface of the thermoformable layer that will contact the user's skin. The mask or other therapeutic device 12 has at least one eye hole 22 and at least one mouth hole 24. This configuration allows for the maximum amount of contact from the cosmetic or therapeutic mask to the user's face. The strapped fabric 16 is applied afterwards and used to hold the formable layer to the user's face. The strapped fabric has a first end 44 and second end 46 each having preferable having a fastener. Fasteners such as hook and loop, buttons, and other known fastening means can be used. The strapped fabric 16 also has at least one eye hole 22 and at least one mouth hole 24.

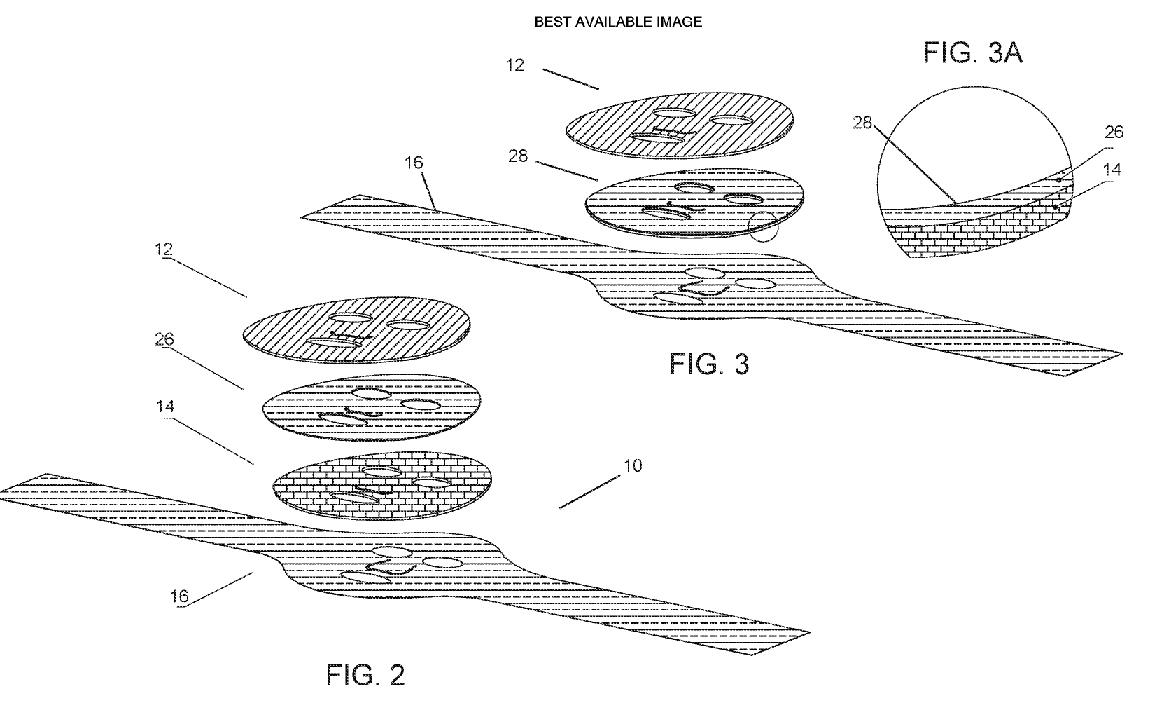

Illustrated in FIG. 2 is an exploded view of another embodiment of the present invention in which a layer of strapless fabric 26 is included in the retainer assembly. As shown in FIG. 3, the thermoformable layer 14 has been bonded to a layer of strapless fabric 26 as a sub-assembly 28 of the retainer 10 forming a composite. The thermoformable layer 14 also has at least one eyehole 22 and at least one mouth hole 24. Shown in FIG. 3A, the strapless fabric 26 is bonded to the thermoformable layer 14 and is exposed towards the user's face with the cosmetic or therapeutic mask there between. The user first places the skin-engaging face 38 of the cosmetic or therapeutic mask 12 on their face and then places the heated sub-assembly 28 on the rear face 40 of cosmetic or therapeutic mask 12. The internal face 42 of the strapped fabric 16 is then placed on top of the thermoformable layer 14 which is then strapped into place using straps 18.

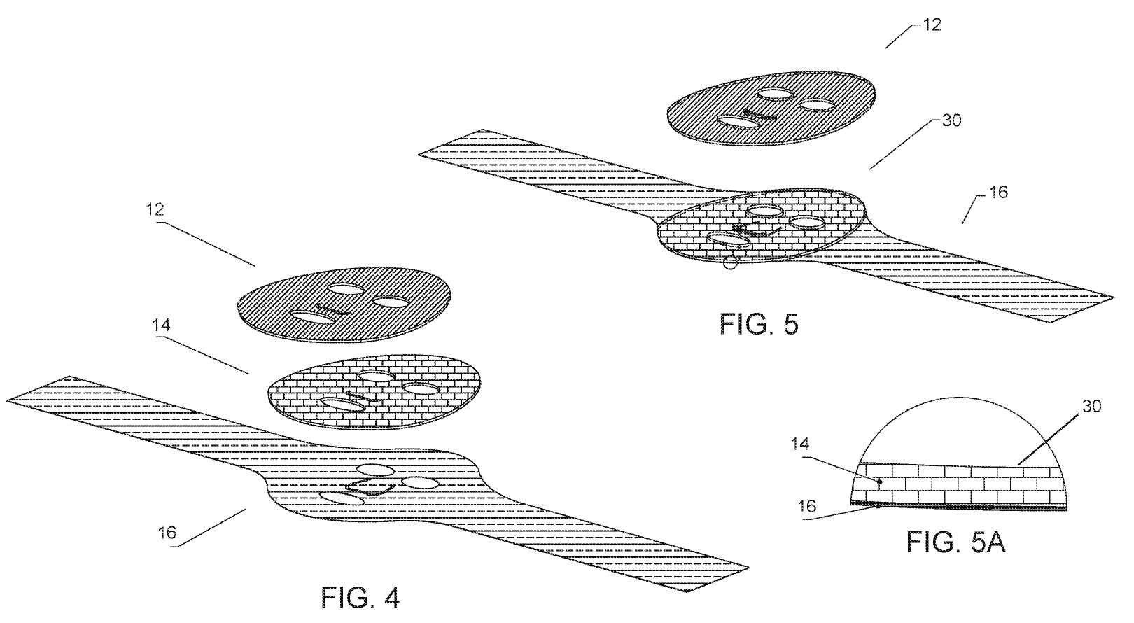

Illustrated in FIGS. 4 and 5 is another embodiment of the present invention in which the thermoformable layer 14 is bonded to the strapped fabric 16 as a sub-assembly 30. The thermoformable layer 14 is left exposed towards the user's face as seen in FIG. 5A. The cosmetic or therapeutic mask 12 is applied to the thermoformable layer and then to the user's face.

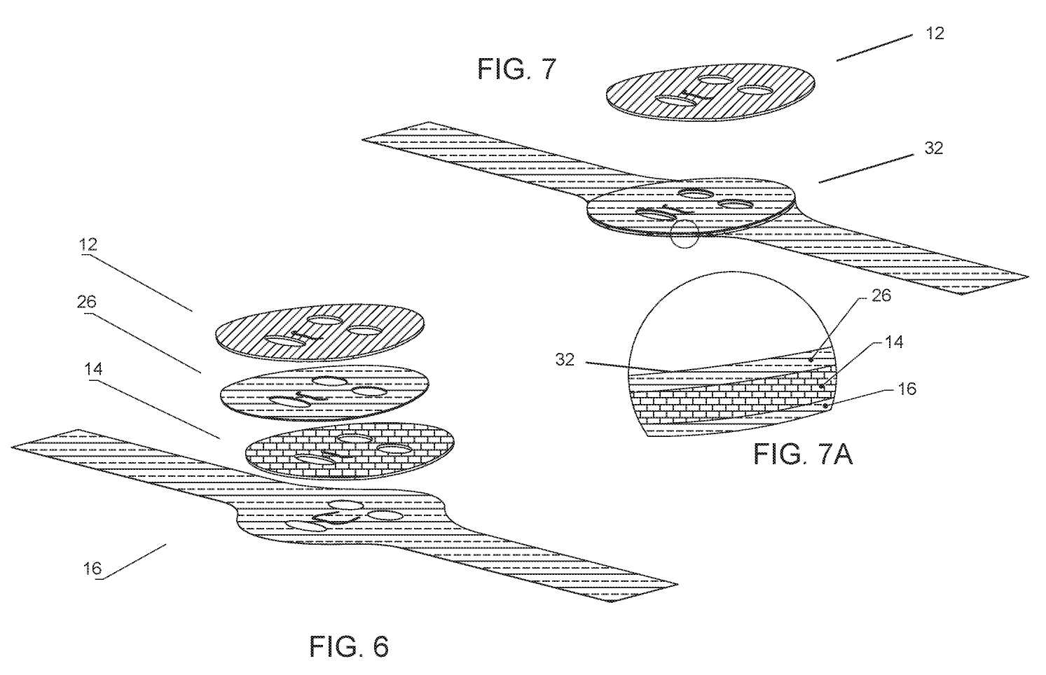

Illustrated in FIGS. 6 and 7 is another embodiment of the present invention in which the thermoformable layer 14 is bonded on one side to strapped fabric 16 and on the other side to strapless fabric 26 as sub-assembly 32. The three component retainer assembly is heated, including strapless fabric 26, thermoformable layer 14, and strapped fabric 16, to the glass transition temperature of the thermoformable layer 14 and then pressed to the users face until the proper geometric contours are achieved. Afterwards, the cosmetic or therapeutic mask 12 is applied to the retainer assembly and applied to the user's face.

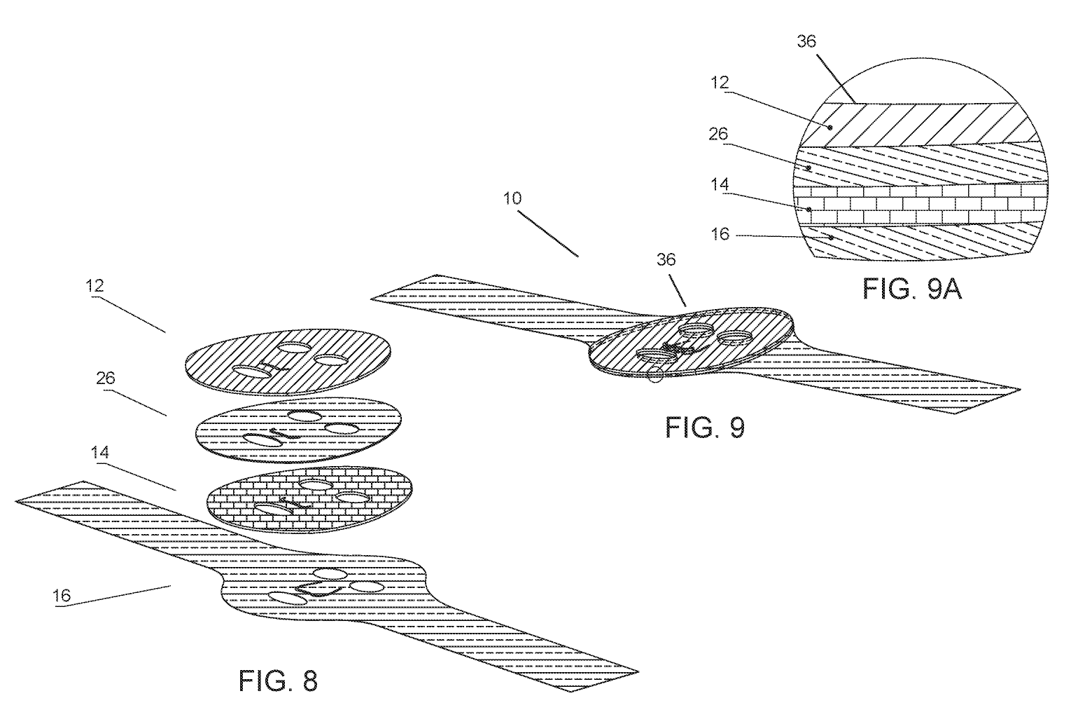

Illustrated in FIGS. 8 and 9 is another embodiment of the present invention in which one side of the thermoformable layer 14 is bonded to strapped fabric 16 and the other is bonded to strapless fabric 26 as sub-assembly 34. In this embodiment a cosmetic or therapeutic mask 12 may be adhered, bonded, or fastened to the remaining surface of the strapless fabric. After heating to the glass transition temperature of the thermoformable material, the retainer assembly is depressed onto the user's face in order to form to the contours of the user's face.

Bonding between layers is generally achieved by heating the thermoformable layer beyond its heat deflection temperature and pressing the layer onto the layer with which it is desired to make a composite material. The layers may also be made into a composite by injecting or casting liquefied polymer onto the layer. It should be understood by those of ordinary skill in the art that similar techniques can be used to bond the various layers of the present invention together.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention.

What is claimed is:

1. A retainer for a cosmetic or therapeutic mask comprising:
a strapped fabric layer;
a thermoformable layer; and
a mask layer.

2. The retainer of claim 1 wherein the thermoformable layer further comprises a polymer able to be molded at a temperature of 60 degrees Celsius or less.

3. The retainer of claim 2 wherein the polymer comprises ethyl vinyl acetate.

4. The retainer of claim 2 wherein the polymer comprises polycaprolactone.

5. The retainer of claim 1 wherein the strapped fabric layer, thermoformable layer, and the mask layer each has at least one eye hole.

6. The retainer of claim 1 wherein the strapped fabric layer, thermoformable layer, and the mask layer each has at least one mouth hole.

7. The retainer of claim 1 wherein the strapped fabric layer further comprises a first end having a first fastener and a second end having a second fastener.

8. A retainer for a cosmetic or therapeutic mask comprising:
a strapped fabric layer;
a thermoformable layer and a strapless fabric layer formed into a composite; and
a mask layer.

9. The retainer of claim 8 wherein the thermoformable layer further comprises a polymer able to be molded at a temperature of 60 degrees Celsius or less.

10. The retainer of claim 9 wherein the polymer comprises ethyl vinyl acetate.

11. The retainer of claim 9 wherein the polymer comprises polycaprolactone.

12. The retainer of claim 8 wherein the strapped fabric layer, composite, and the mask layer each has at least one eye hole.

13. The retainer of claim 8 wherein the strapped fabric layer, composite, and the mask layer each has at least one mouth hole.

14. The retainer of claim 8 wherein the strapped fabric layer further comprises a first end having a first fastener and a second end having a second fastener.

15. A retainer for a cosmetic or therapeutic mask comprising:
a strapped fabric layer and a thermoformable layer formed into a composite; and
a mask layer.

16. The retainer of claim 15 wherein the thermoformable layer further comprises a polymer able to be molded at a temperature of 60 degrees Celsius or less.

17. The retainer of claim 16 wherein the polymer comprises ethyl vinyl acetate.

18. The retainer of claim 16 wherein the polymer comprises polycaprolactone.

19. The retainer of claim 15 wherein the composite and the mask layer each has at least one eye hole.

20. The retainer of claim 15 wherein the composite and the mask layer each has at least one mouth hole.

21. The retainer of claim 15 wherein the strapped fabric layer further comprises a first end having a first fastener and a second end having a second fastener.

22. A retainer for a cosmetic or therapeutic mask comprising:
a strapped fabric layer, a thermoformable layer, and a strapless fabric layer formed into a composite; and
a mask layer.

23. The retainer of claim 22 wherein the thermoformable layer further comprises a polymer able to be molded at a temperature of 60 degrees Celsius or less.

24. The retainer of claim 23 wherein the polymer comprises ethyl vinyl acetate.

25. The retainer of claim 23 wherein the polymer comprises polycaprolactone.

26. The retainer of claim 22 wherein the composite and the mask layer each has at least one eye hole.

27. The retainer of claim 22 wherein the composite and the mask layer each has at least one mouth hole.

28. The retainer of claim 22 wherein the strapped fabric layer further comprises a first end having a first fastener and a second end having a second fastener.

29. A retainer for a cosmetic or therapeutic mask comprising a strapped fabric layer, a thermoformable layer, a strapless fabric layer, and a mask layer formed into a composite.

30. The retainer of claim 29 wherein the thermoformable layer further comprises a polymer able to be molded at a temperature of 60 degrees Celsius or less.

31. The retainer of claim 30 wherein the polymer comprises ethyl vinyl acetate.

32. The retainer of claim 30 wherein the polymer comprises polycaprolactone.

33. The retainer of claim 29 wherein the composite has at least one eye hole.

34. The retainer of claim 29 wherein the composite has at least one mouth hole.

35. The retainer of claim 29 wherein the strapped fabric layer further comprises a first end having a first fastener and a second end having a second fastener.
